(19) United States
(12) Patent Application Publication
(10) Pub. No.: US 2009/0012104 A1
(43) Pub. Date: Jan. 8, 2009

(54) Inhibitors of 5'-Methylthioadenosine Phosphorylase and 5'-Methylthioadenosine/S-Adenosylhomocysteine Nucleosidase

(76) Inventors: Yarlagadda S. Babu, Birmingham, AL (US); Vivekanand P. Kamath, Birmingham, AL (US); Philip E. Morris, JR., Birmingham, AL (US)

Correspondence Address: Bradley Arant Rose & White, 1819 Fifth Avenue North, Birmingham, AL, 35203-2104, US

(21) Appl. No.: 11/658,794

(22) PCT Filed: Jul. 26, 2005
(86) PCT No.: PCT/US05/26419

§ 371 Date: Jan. 25, 2007

Related U.S. Application Data
(60) Provisional application No. 60/591,442, filed on Jul. 27, 2004.
(60) Provisional application No. 60/619,126, filed on Oct. 15, 2004.

(51) Int. Cl. A61K 31/519; C07D 487/04; A61P 35/02

(52) U.S. Cl. 514/265.1; 544/280

(57) ABSTRACT

Described are compounds of the general formula (I), VI and (VII). Also described are pharmaceutical compositions comprising the compounds identified. The compounds and pharmaceutical compositions described are inhibitors of 5′-methylthioadenosine/S-adenosylhomocystein nucleosidase (MTAN) and/or 5′-methylthioadenosine phosphorylase (MTAP). Methods of treatment using the compounds and pharmaceutical compositions described are also provided for preventing and/or treating disease states and/or conditions by inhibiting MTAN and/or MTAP in patients.

This application claims priority to and benefit of U.S. Provisional Patent Application Nos. 60/591,442 (filed Jul. 27, 2004) and 60/619,126 (filed Oct. 15, 2004).

### FIELD OF THE DISCLOSURE

The present disclosure relates to certain compounds that are inhibitors of 5′-methylthioadenosine/S-adenosylhomocysteine nucleosidase (MTAN) and/or 5′-methylthioadenosine phosphorylase (MTAP). The present disclosure also relates to pharmaceutical compositions comprising the MTAP and MTAN inhibitors disclosed herein, as well as methods of using the inhibitors to treat various diseases states/conditions and methods for producing the inhibitors disclosed herein.

### BACKGROUND

The present disclosure describes the identification of compounds which are inhibitors of 5′-methylthioadenosine/S-adenosylhomocysteine nucleosidase (referred to in this disclosure as MTAN). MTAN catalyzes the hydrolysis of 5′methylthioadenosine (MTA) to adenine and 5′-methylthioribose (MTR) and the hydrolysis of S-adenosylhomocysteine (SAH) to adenine and S-ribosylhomocysteine (SRH). MTAN occurs in a variety of bacterial cell types (both Gram positive and Gram negative). MTAN is not found in eukaryotic cell types, including humans.

Inhibition of MTAN may have several important effects. First, MTA is produced as a by product during the formation of spermadine by the action of spermadine synthase. MTA is a potent inhibitor of spermadine synthase. Therefore, the buildup of MTA, which may occur as a result of MTAN inhibition, may result in decreased polyamine biosynthesis. Polyamines are postulated to play key roles in growth processes and the regulation of DNA synthesis. Therefore, inhibition of MTAN by the compounds disclosed may impact the regulation of cell growth and/or DNA synthesis.

Second, MTAN inhibition would block the methionine salvage pathway in bacterial cells. In the bacterial methionine salvage pathway, MTA is converted to MTR by MTAN. MTR is then acted on by a pathway of bacterial enzymes to produce methionine and S-adenosylmethionine. S-adenosylmethionine is an important methyl donor in a variety of intracellular reactions (yielding the product SAH, which is also a substrate of MTAN). Inhibiting the conversion of MTA will block methionine salvage and also impact reactions dependent on methyl transfer from S-adenosylmethionine.

Finally, MTAN inhibition may impact the production of various autoinducer (AI) molecules important for a variety of bacterial functions. AI molecules are involved in a bacterial process termed “quorum sensing”, through which bacteria monitor the presence of other bacteria in their surroundings by producing and responding to various AI molecules. In this manner, bacteria can determine a count of other bacteria in their surroundings and modulate their responses accordingly. A variety of behaviors are controlled by this quorum sensing pathway. These behaviors are generally those that require a group of bacteria to carry out the behavior in synchrony to be effective and include, but are not limited to, bioluminescence, expression of virulence factors, biofilm formation, sporulation, conjugation and pigment production. In essence, quorum sensing allows individual bacteria to function as a group and resemble multicellular organisms. This communication is both species specific (for the AI-type 1 pathway) and species non-specific (for the AI-type 2 pathway). MTAN is involved in the regulation of both the AI-1 and AI-2 pathways.

Various quorum sensing pathways are present in different bacteria. Gram-negative bacteria employ the paradigm LuxIR circuits, while Gram-positive bacteria employ the paradigm oligopeptide, two-component circuits. In the LuxIR paradigm, a LuxI type protein catalyzes the formation of a specific acyl-homoserine lactone (AHL) Al (referred to as an AI-type 1 autoinducer) by transferring a specific acyl side chain from an acyl-ACP to the homocysteine moiety of S-adenosylmethionine (SAM), with the production of MTA as a by product. The AHL can freely diffuse in and out of bacterial cells, and will therefore, increase within a bacterial cell as the population of bacterial cells increases. The AHL AI binds to a specific LuxR type protein when the concentration of the AHL reaches a threshold level. The LuxR-AHL complex then activates the transcription of a certain set of quorum sensing regulated genes through interaction with quorum sensing regulated promoters. The specificity of the quorum sensing pathway is exquisite and bacterial species only respond to the AHL AI produced by the same species.

MTA, which is produced as a by-product of AHL production, is toxic and must be removed from the cell. As discussed above, MTAN catalyzes the breakdown of MTA into adenine and MTR. Therefore, by inhibiting MTAN, MTA levels will increase leading to a bactericidal or bacteriostatic effect.

MTAN is also involved in the species-independent quorum signaling pathway, or the AI-type 2 pathway. Contrary to the AI-type 1 pathway, the AI-type 2 pathway allows for communication between different species of bacteria, allowing interspecies cellular communication. Also unlike the AI-type 1 pathway, the AI-type 2 pathway is present in both Gram-positive and Gram-negative cell types. The AI molecule produced by the AI-type 2 pathway (referred to as AI-2) is the same in all species of bacteria characterized to date. LuxS cleaves SRH to produce AI-2 and homocysteine. SRH is produced by the degradation of SAH by MTAN to adenine and SRH. SAH is produced as a by-product of methylation reactions utilizing S-adenosylmethionine (SAM), and must be rapidly cleared as SAH is an inhibitor of SAM-dependent methyl transferases. Therefore, inhibition of MTAN would inhibit both the production of AI-2 by decreasing the availability of the LuxS substrate SRH and inhibit SAM-dependent methylation reactions via the accumulation of SAH and the consequent negative regulation of SAH on the SAM-dependent methyl transferases.

As a result, MTAN is an important target for the development of novel anti-microbial agents. Such new anti-microbial agents may provide alternate treatment to recently reported “super-bugs” that are resistant to even the most powerful of the currently used antibiotics. Since MTAN is not present in humans, this new class of MTAN inhibitors would not be expected to harm the host.

The present disclosure describes the identification of a series of inhibitors of MTAN. In addition, the present disclosure describes pharmaceutical compositions comprising the MTAN inhibitors disclosed herein, as well as methods of using the inhibitors to treat various diseases states and/or conditions and methods for producing the inhibitors disclosed herein.

MTAN shares certain structural features with 5′-methylthioadenosine phosphorylase (MTAP). MTAP is an enzyme found in a variety of organisms, including humans, and catalyzes the reversible phosphorolysis of MTA to adenine and 5′-methylthioribose-1-phosphate (MTR-IP). Both MTAN and MTAP have active sites that can be divided into three discrete regions: (i) the adenine/purine binding region; (ii) the ribose binding region; and (iii) the 5′-alkylthio binding region. While MTAN and MTAP possess certain similarities, there are also dissimilarities. For example, the ribose binding site of MTAN lacks the amino acid contacts to coordinate a phosphate anion. As a result, MTAN is a nucleosidase rather than a reversible phosphorylase. In addition, the 5′-alkylhio binding site is somewhat more extended in MTAN than in MTAP.

As discussed above, MTA is produced as a by-product during the formation of spermadine by the action of spermadine synthase. MTA is a potent inhibitor of spermadine synthase. Therefore, the buildup of MTA, which may occur as a result of MTAP inhibition, may result in decreased polyamine biosynthesis. Polyamines are postulated to play key roles in growth processes and the regulation of DNA synthesis. In addition, this salvage reaction is the principal source of free adenine in human cells. Because of its importance in coupling the purine salvage pathway to polyamine biosynthesis, MTAP is a potential chemotherapeutic target. Indeed, cancer cell lines lacking MTAP do display increased sensitivity towards known chemotherapeutic drugs such as methotrexate and azaserine in the presence of MTA, whereas cancer cell lines with MTAP activity are not as severely affected. In view of these observations, the treatment of MTAP⁺ tumors may be enhanced by the co-administration of a potent MTAP inhibitor together with traditional chemotherapeutic compounds that specifically target the de novo purine biosynthetic machinery.

Therefore, the present disclosure also describes the identification of a series of inhibitors of MTAP. In addition, the present disclosure describes pharmaceutical compositions comprising the MTAP inhibitors disclosed herein, as well as methods of using the inhibitors to treat various diseases states and/or conditions and methods for producing the inhibitors disclosed herein.

### BRIEF DESCRIPTION OF THE FIGURES

FIG. 1 shows one embodiment of a general synthetic scheme for 2-Amino-4-[5-(4-amino-5H-pyrrolo[3,2-d]pyrimidin-7-yl)-3,4-dihydroxy-pyrrolidin-2-ylmethylsulfanyl]-butyric acid.

### DETAILED DESCRIPTION

This present disclosure provides compounds of the general formula (I), (VI) and (VII), or pharmaceutically acceptable salts thereof, or esters thereof, or prodrugs thereof and tautomers and polymorphic variants of any of the foregoing. All sugars described herein may either be in D or L configuration.

With regard to compounds having the general formula (I),

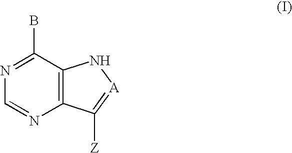

A is selected from the group consisting of N and CD,
where D is selected from the group consisting of: H, halogen, unsubstituted alkyl, substituted alkyl, optionally substituted aryl, optionally substituted heterocycle, optionally substituted cycloalkyl, OH, NH₂, NHR₁, NR₁R₂ and SR₃,
   where R₁, R₂ and R₃ are each independently selected from the group consisting of: H, unsubstituted alkyl, substituted alkyl, optionally substituted heterocycle, optionally substituted cycloalkyl and optionally substituted aryl;
      B is selected from the group consisting of NH₂ and NHR₄,
where R₄ is selected from the group consisting of: H, unsubstituted alkyl, substituted alkyl, optionally substituted heterocycle, optionally substituted cycloalkyl and optionally substituted aryl; and
   Z is selected from the group consisting of compound (II), compound (IV), and compound (V),

where

compound II is:

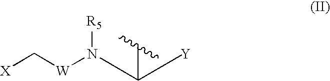

wherein
X is selected from the group consisting of compound (III), R₆S, H, unsubstituted alkyl, substituted alkyl, optionally substituted heterocycle, optionally substituted cycloalkyl and optionally substituted aryl;
   where R₆ is selected from the group consisting of H, unsubstituted alkyl, substituted alkyl, optionally substituted heterocycle, optionally substituted cycloalkyl and optionally substituted aryl; and

Compound (III) is:

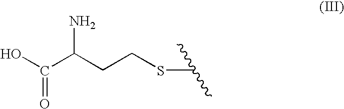

wherein

W is selected from the group consisting of CHR₇ and CR₇%,
where R₇ and R₈ are each independently selected from the group consisting of H, unsubstituted alkyl, substituted alkyl, optionally substituted heterocycle, optionally substituted cycloalkyl and optionally substituted aryl; and

Y is selected from the group consisting of H and CH₂R₉;
where R₉ is selected from the group consisting of H, unsubstituted alkyl, substituted alkyl, optionally substituted heterocycle, optionally substituted cycloalkyl and optionally substituted aryl; and

R₅ is selected from the group consisting of H, unsubstituted alkyl, substituted alkyl, optionally substituted heterocycle, optionally substituted cycloalkyl and optionally substituted aryl;

where

compound (IV) is,

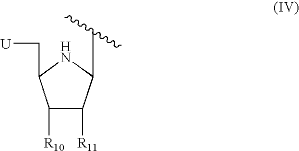

where U is selected from the group consisting of compound (III), R₁₂S, H, unsubstituted alkyl, substituted alkyl, optionally substituted heterocycle, optionally substituted cycloalkyl and optionally substituted aryl;
   where R₁₂ is selected from the group consisting of H, unsubstituted alkyl, substituted alkyl, optionally substituted heterocycle, optionally substituted cycloalkyl and optionally substituted aryl; and
   where compound (III) is as described above in relation to compound (I), including all substituents of compound (III); and
   where R₁₀ and R₁₁ are each selected from the group consisting of H, OH and halogen; and

where

compound (V) is

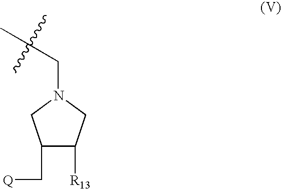

where Q is selected from the group consisting of compound (III), R₁₄S, H, unsubstituted alkyl, unsubstituted alkyl, optionally substituted heterocycle, optionally substituted cycloalkyl and optionally substituted aryl;
where R₁₄ is selected from the group consisting of H, unsubstituted alkyl, substituted alkyl, optionally substituted heterocycle, optionally substituted cycloalkyl and optionally substituted aryl; and
where compound (III) is as described above in relation to compound (I), including all substituents of compound (III); and
where R₁₃ is selected from the group consisting of: H, OH and halogen.
   With regard to compounds having the general formula (VI),

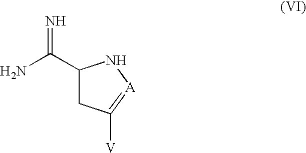

A is selected from the group consisting of N and CD,
where N and CD are as described above in relation to compound (I), including all substituents of N and CD; and
   V is selected from the group consisting of compound (II), compound (IV), and compound (V),
where compound (II), compound (IV), and compound (V) are as described above in relation to compound (I), including all substituents of compounds (II), (IV), and (V).
   With regard to compounds having the general formula (VII)

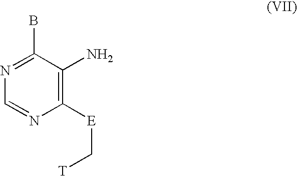

where B is selected from the group consisting of NH₂ and NHR₄,
where NH₂ and NHR₄ are as described above in relation to compound (I), including all substituents of NH₂ and NHR₄; and
   E is selected from the group consisting of: O, CHR₁₅, CR₁₅R₁₆, and N,

where R₁₅ and R₁₆ are each independently selected from the group consisting of H, unsubstituted alkyl, substituted alkyl, optionally substituted heterocycle, optionally substituted cycloalkyl and optionally substituted aryl; and

T is selected from the group consisting of compound (II), compound (IV), and compound (V),

where compound (II), compound (IV), and compound (V) are as described above in relation to compound (I), including all substituents of compounds (II), (IV), and (V).

### Exemplary Structures

Structures encompassed by the present disclosure include, but are not limited to, the structures illustrated below, with the substituent groups being as described in the instant specification.

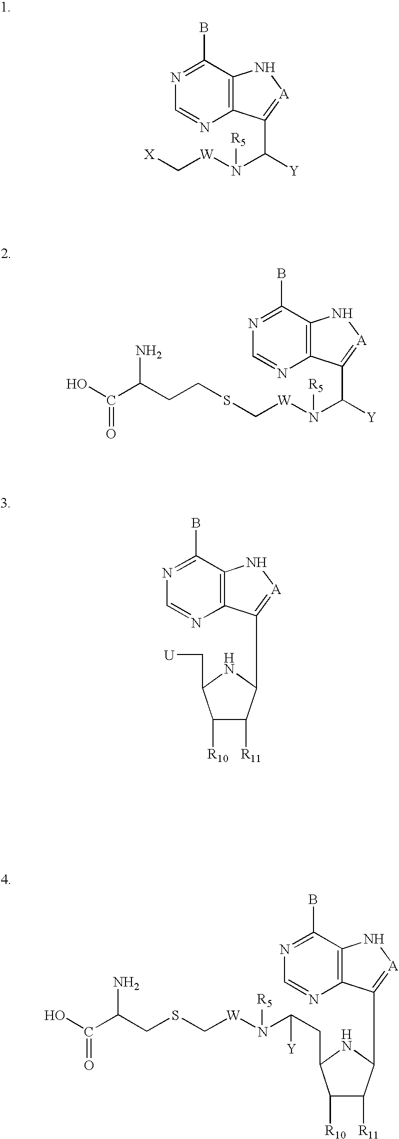

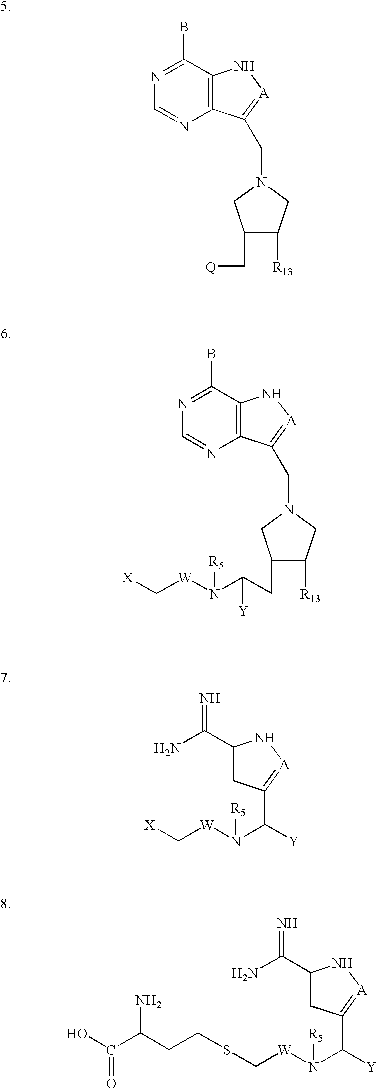

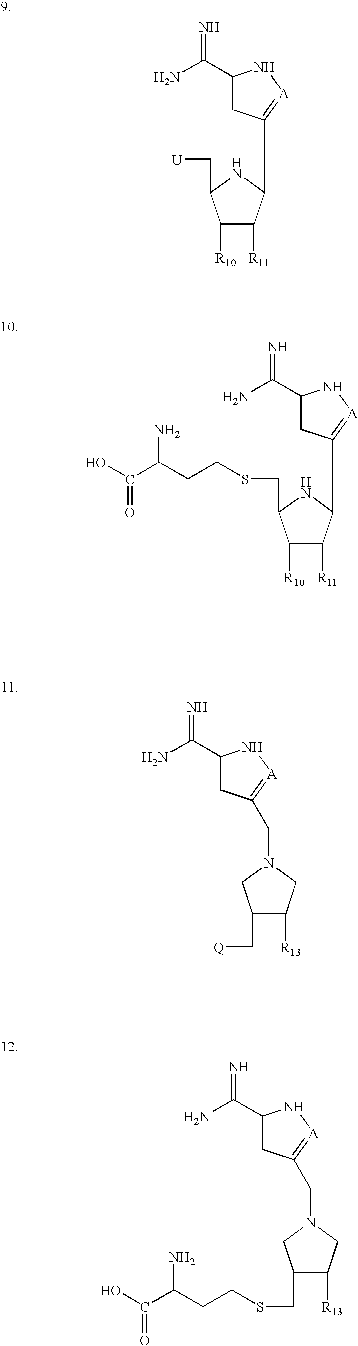

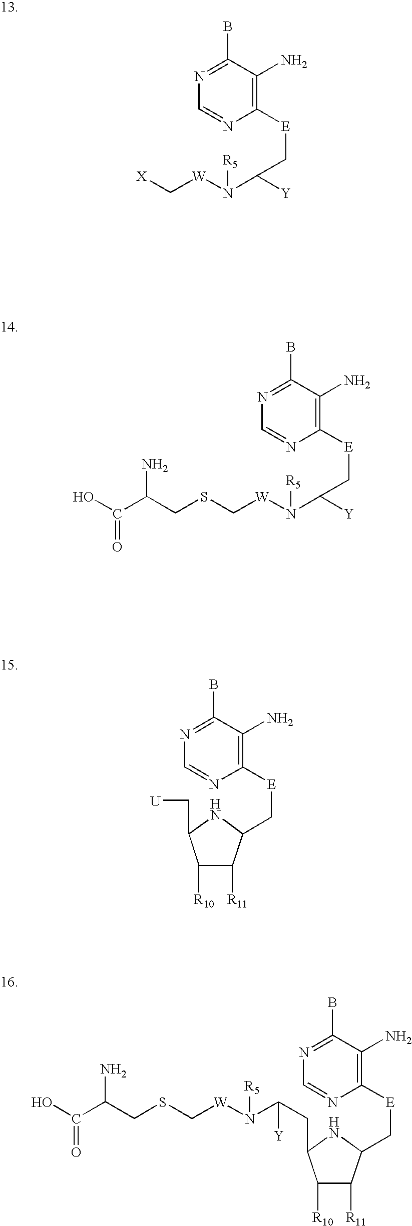

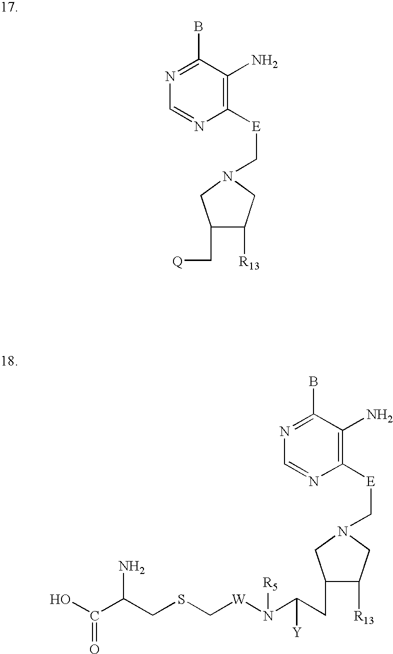

### DEFINITIONS

As used in this specification, the followings words and phrases have the meanings as defined below, unless otherwise limited in specific instances, either individually or as part of a larger group.

As used herein, the term “alkyl” whether used alone or as part of a substituent group, includes saturated straight chain carbon groups and branched chain isomers of the straight chain carbon groups, as well as those straight or branched chain carbons groups containing at least one double bond between two carbon atoms thereof and those containing at least one triple bond between two carbon atoms thereof.

The term “unsubstituted alkyl” refers to alkyl groups that do not contain heteroatoms. Thus the phrase includes straight chain alkyl groups such as methyl, ethyl, propyl, butyl, pentyl, hexyl, heptyl, octyl, nonyl, decyl, undecyl, dodecyl and the like, as well as branched chain isomers of straight chain alkyl groups, as well as corresponding compounds with carbon-carbon double bonds and carbon-carbon triple bonds. The phrase unsubstituted alkyl groups includes primary alkyl groups, secondary alkyl groups, and tertiary alkyl groups. Unsubstituted alkyl groups may be bonded to one or more carbon atom(s), oxygen atom(s), nitrogen atom(s), and/or sulfur atom(s) in the parent compound. Preferred unsubstituted alkyl groups include straight and branched chain alkyl groups and cyclic alkyl groups having 1 to 20 carbon atoms, from 1 to 10 carbon atoms or from 1 to 6 carbon atoms.

The term “substituted alkyl” refers to an unsubstituted alkyl group as defined above in which one or more bonds to a carbon(s) or hydrogen(s) are replaced by a bond to non-hydrogen or non-carbon atoms. The position of substitution of the non-carbon atom may be any position on the alkyl group and may be at multiple positions on the alkyl group. The non-carbon atoms include but not limited to, a halogen atom in halides such as, but not limited to, F, Cl, Br, and I; and cyclic groups such as, but not limited to, cycloalkyl, aryl or heterocyclo; and oxygen atom in groups such, but not limited to, as hydroxyl groups, alkoxy groups, aryloxy groups, and ester groups; a sulfur atom in groups such as, but not limited to, thiol groups, alkyl and aryl sulfide groups, sulfone groups, sulfonyl groups, and sulfoxide groups; and nitrogen atom in groups such as, but not limited to, amines, amides, alkylamines, dialkylamines, arylamines, alkylarylamines, diarylamines, N-oxides, imides, and enamines; and other heteroatoms in various other groups.

The term “cycloalkyl” refers to an optionally substituted, saturated or unsaturated cyclic hydrocarbon ring systems, such as those containing 1 to 3 rings and 3 to 7 carbons per ring. Exemplary groups include cyclopropyl, cyclobutyl, cyclopentyl, cyclohexyl, cycloheptyl, cyclooctyl, cyclodecyl, cyclododecyl and adamantyl. Exemplary substituents include one or more alkyl or substituted alkyl groups as described above, or one or more groups described above in the definition of substituted alkyl.

The terms “heterocyclyl”, “heterocyclic” and “heterocyclo” refer to an optionally substituted, saturated or unsaturated, aromatic or non-aromatic cyclic group (which may be monocyclic, bicyclic or tricyclic), which has at least one heteroatom (such as, but not limited to, N, O or S) in at least one carbon-containing ring. Each ring of the heterocyclic group containing a heteroatom may have 1, 2 or 3 heteroatoms selected from nitrogen atoms, oxygen atoms, and sulfur atoms, where the nitrogen and sulfur heteroatoms may also optionally be oxidized and the nitrogen heteroatoms may also optionally be quaternized. The heterocyclic group may be attached at any heteroatom or carbon atoms. Exemplary substituents include one or more alkyl or substituted alkyl groups as described above or one or more groups described above in the definition of substituted alkyl.

The term “aryl,” refers to an optionally substituted aromatic hydrocarbon which can be a single ring or multiple rings which are fused together or linked covalently, such as, but not limited to, phenyl, naphthyl, biphenyl and diphenyl groups. Exemplary substituents include one or more alkyl or substituted alkyl groups as described above, or one or more groups described above in the definition of substituted alkyl.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, oxalic, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The term “prodrug” is meant to include functional derivatives of the compounds disclosed which are readily convertible in vivo into the required compound. Thus, in the methods of treatment of the present disclosure, the term “administering” shall encompass the treatment of the various disease states/conditions described with the compound specifically disclosed or with a prodrug which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

The terms “prevent”, “preventing”, “prevention” “suppress”, “suppressing” and suppression as used herein refer to administering a compound prior to the onset of clinical symptoms of a disease state/condition so as to prevent any symptom, aspect or characteristic of the disease state/condition. Such preventing and suppressing need not be absolute to be useful.

The terms “treat”, “treating” and treatment as used herein refers to administering a compound after the onset of clinical symptoms of a disease state/condition so as to reduce or eliminate any symptom, aspect or characteristic of the disease state/condition. Such treating need not be absolute to be useful.

The term “in need of treatment” as used herein refers to a judgment made by a caregiver that a patient requires or will benefit from treatment. This judgment is made based on a variety of factors that are in the realm of a caregiver's expertise, and may include the knowledge that the patient is ill as the result of a disease state/condition that is treatable by a compound or pharmaceutical composition of the disclosure.

The term “in need of prevention” as used herein refers to a judgment made by a caregiver that a patient requires or will benefit from prevention. This judgment is made based on a variety of factors that are in the realm of a caregiver's expertise, and may include the knowledge that the patient may become ill as the result of a disease state/condition that is treatable by a compound or pharmaceutical composition of the disclosure.

The term “individual” or “patient” as used herein refers to any animal, including mammals, such as, but not limited to, mice, rats, other rodents, rabbits, dogs, cats, swine, cattle, sheep, horses, or primates, or humans. The term may specify male or female or both, or exclude male or female; and

The term “therapeutically effective amount”, in reference to the treating, preventing or suppressing of a disease state/condition, refers to an amount of a compound either alone or as contained in a pharmaceutical composition that is capable of having any detectable, positive effect on any symptom, aspect, or characteristics of the disease state/condition. Such effect need not be absolute to be beneficial.

### SELECTED EMBODIMENTS OF THE DISCLOSURE

Certain selected embodiments of the compounds of the present disclosure will now be provided. These examples are not meant to limit the breadth of the compounds as disclosed and claimed, but are provided as examples of certain compounds that fall within the scope of the present disclosure.

In one embodiment, a compound of the present disclosure having the general formula (I) is described where B is NH₂, A is N or CH, and Z is compound (II). In one embodiment when Z is compound (II), W is CR₇R₉ with R₇ and R₉ being a H or a C₁-C₅ unsubstituted alkyl or a C₁-C₅ substituted alkyl, R₅ is a H or a C₁-C₅ unsubstituted alkyl or a C₁-C₅ substituted alkyl, and Y is CH₂R₉, with R₉ being a H or a C₁-C₅ unsubstituted alkyl or a C₁-C₅ substituted alkyl, X is phenyl, 3-chlorophenyl, 4-chlorophenyl, 4-fluorophenyl, 3-methylphenyl, 4-methylphenyl, benzyl, hydroxyethyl, fluoroethyl, naphthyl, methyl and ethyl, specifically, X may be phenyl, 3-methylphenyl or 4-chlorophenyl. In an alternate embodiment when Z is compound (II), W is CR₇R₈ with R₇ and R₈ being a H or a C₁-C₅ unsubstituted alkyl or a C₁-C₅ substituted alkyl, R₅ is a H or a C₁-C₅ unsubstituted alkyl or a C₁-C₅ substituted alkyl, and Y is CH₂R₉, with R₉ being a H or a C₁-C₅ unsubstituted alkyl or a C₁-C₅ substituted alkyl, X is R₆S, with R₆ being a H or a C₁-C₅ unsubstituted alkyl or a C₁-C₅ substituted alkyl, specifically R₆ may be CH₃ or CH₂CH₃. In yet another alternate embodiment when Z is compound (II), W is CR₇R₈ with R₇ and R₈ being a H or a C₁-C₅ unsubstituted alkyl or a C₁-C₅ substituted alkyl, R₅ is a H or a C₁-C₅ unsubstituted alkyl or a C₁-C₅ substituted alkyl, and Y is CH₂R₉, with R₉ being a H or a C₁-C₅ unsubstituted alkyl or a C₁-C₅ substituted alkyl, X is H or a C₁-C₅ unsubstituted alkyl or a C₁-C₅ substituted alkyl, specifically, X may be CH₃ or CH₂CH₃. In still another embodiment when Z is compound (II), W is CR₇R₈ with R₇ and R₈ being a H or a C₁-C₅ unsubstituted alkyl or a C₁-C₅ substituted alkyl, R₅ is a H or a C₁-C₅ unsubstituted alkyl or a C₁-C₅ substituted alkyl, and Y is CH₂R₉, with R₉ being a H or a C₁-C₅ alkyl or a C₁-C₅ substituted alkyl, X is compound (II).

In one embodiment, a compound of the present disclosure having the general formula (I) is described where B is NH₂, A is N or CH, and Z is compound (IV). In one embodiment where Z is compound (IV), R₁₀ and R₁₁, are each independently H, OH or halogen, U is phenyl, 3-chlorophenyl, 4-chlorophenyl, 4-fluorophenyl, 3-methylphenyl, 4-methylphenyl, benzyl, hydroxyethyl, fluoroethyl, naphthyl, methyl and ethyl, specifically, U may be phenyl, 3-methylphenyl or 4-chlorophenyl. In one embodiment where Z is compound (IV), R₁₀ and R₁₁ are each independently H, OH or halogen, U is SR₁₂, with R₁₂ being a H or a C₁-C₅ unsubstituted alkyl or a C₁-C₅ substituted alkyl, specifically R₁₂ may be CH₃ or CH₂CH₃. In one embodiment where Z is compound (IV), R₁₀ and R₁₁ are each independently H, OH or halogen, U is H or a C₁-C₅ unsubstituted alkyl or a C₁-C₅ substituted alkyl, specifically, U may be CH₃ or CH₂CH₃. In one embodiment where Z is compound (IV), R₁₀ and R₁₁ are each independently H, OH or halogen, U is compound (III).

In one embodiment, a compound of the present disclosure having the general formula (I) is described where B is NH₂, A is N or CH, and Z is compound (V). In one embodiment where Z is compound (V), R₁₃ is independently H, OH or halogen, Q is phenyl, 3-chlorophenyl, 4-chlorophenyl, 4-fluorophenyl, 3-methylphenyl, 4-methylphenyl, benzyl, hydroxyethyl, fluoroethyl, naphthyl, methyl and ethyl, specifically, Q may be phenyl, 3-methylphenyl or 4-chlorophenyl. In one embodiment where Z is compound (V), R₁₃ is independently H, OH or halogen, Q is SR₁₄, with R₁₄ being a H or a C₁-C₅ unsubstituted alkyl or a C₁-C₅ substituted alkyl, specifically R₁₄ may be CH₃ or CH₂CH₃. In one embodiment where Z is compound (V), R₁₃ is independently H, OH or halogen, Q is H or a C₁-C₅ unsubstituted alkyl or a C₁-C₅ substituted alkyl, specifically, Q may be CH₃ or CH₂CH₃. In one embodiment where Z is compound (V), R₁₃ is independently H, OH or halogen, Q is compound (III).

In one embodiment, a compound of the present disclosure having the general formula (VI) is described where A is N or CH and V is compound (II). In one embodiment when V is compound (II), W is CR₇R₈ with R₇ and R₈ being a H or a C₁-C₅ unsubstituted alkyl or a C₁-C₅ substituted alkyl, R₅ is a H or a C₁-C₅ unsubstituted alkyl or a C₁-C₅ substituted alkyl, and Y is CH₂R₉, with R₉ being a H or a C₁-C₅ unsubstituted alkyl or a C₁-C₅ substituted alkyl, X is phenyl, 3-chlorophenyl, 4-chlorophenyl, 4-fluorophenyl, 3-methylphenyl, 4-methylphenyl, benzyl, hydroxyethyl, fluoroethyl, naphthyl, methyl and ethyl, specifically, X may be phenyl, 3-methylphenyl or 4-chlorophenyl. In an alternate embodiment when Z is compound (II), W is CR₇R₉ with R₇ and R₉ being a H or a C₁-C₅ unsubstituted alkyl or a C₁-C₅ substituted alkyl, R₅ is a H or a C₁-C₅ unsubstituted alkyl or a C₁-C₅ substituted alkyl, and Y is CH₂R₉, with R₉ being a H or a C₁-C₅ unsubstituted alkyl or a C₁-C₅ substituted alkyl, X is R₆S, with R₆ being a H or a C₁-C₅ unsubstituted alkyl or a C₁-C₅ substituted alkyl, specifically R₆ may be CH₃ or CH₂CH₃. In yet another alternate embodiment when Z is compound (II), W is CR₇R₈ with R₇ and R₈ being a H or a C₁-C₅ unsubstituted alkyl or a C₁-C₅ substituted alkyl, R₅ is a H or a C₁-C₅ unsubstituted alkyl or a C₁-C₅ substituted alkyl, and Y is CH₂R₉, with R₉ being a H or a C₁-C₅ unsubstituted alkyl or a C₁-C₅ substituted alkyl, X is H or a C₁-C₅ unsubstituted alkyl or a C₁-C₅ substituted alkyl, specifically, X may be CH₃ or CH₂CH₃. In still another embodiment when Z is compound (II), W is CR₇R₈ with R₇ and R₈ being a H or a C₁-C₅ unsubstituted alkyl or a C₁-C₅ substituted alkyl, R₅ is a H or a C₁-C₅ unsubstituted alkyl or a C₁-C₅ substituted alkyl, and Y is CH₂R₉, with R₉ being a H or a C₁-C₅ alkyl or a C₁-C₅ substituted alkyl, X is compound (III).

In one embodiment, a compound of the present disclosure having the general formula (VI) is described where A is N or CH, and V is compound (IV). In one embodiment when V is compound (IV), R₁₀ and R₁₁ are each independently H, OH or halogen, U is phenyl, 3-chlorophenyl, 4-chlorophenyl, 4-fluorophenyl, 3-methylphenyl, 4-methylphenyl, benzyl, hydroxyethyl, fluoroethyl, naphthyl, methyl and ethyl, specifically, U may be phenyl, 3-methylphenyl or 4-chlorophenyl. In one embodiment where Z is compound (IV), R₁₀ and R₁, are each independently H, OH or halogen, U is SR₁₂, with R₁₂ being a H or a C₁-C₅ unsubstituted alkyl or a C₁-C₅ substituted alkyl, specifically R₁₂ may be CH₃ or CH₂CH₃. In one embodiment where Z is compound (IV), R₁₀ and R₁ are each independently H, OH or halogen, U is H or a C₁-C₅ unsubstituted alkyl or a C₁-C₅ substituted alkyl, specifically, U may be CH₃ or CH₂CH₃. In one embodiment where Z is compound (IV), R₁₀ and R₁₁ are each independently H, OH or halogen, U is compound (III).

In one embodiment, a compound of the present disclosure having the general formula (VI) is described where A is N or CH, and V is compound (V). In one embodiment when V is compound (V), R₁₃ is independently H, OH or halogen, Q is phenyl, 3-chlorophenyl, 4-chlorophenyl, 4-fluorophenyl, 3-methylphenyl, 4-methylphenyl, benzyl, hydroxyethyl, fluoroethyl, naphthyl, methyl and ethyl, specifically, Q may be phenyl, 3-methylphenyl or 4chlorophenyl. In one embodiment where Z is compound (V), R₁₃ is independently H, OH or halogen, Q is SR₁₄, with R₁₄ being a H or a C₁-C₅ unsubstituted alkyl or a C₁-C₅ substituted alkyl, specifically R₁₄ may be CH₃ or CH₂CH₃. In one embodiment where Z is compound (V), R₁₃ is independently H, OH or halogen, Q is H or a C₁-C₅ unsubstituted alkyl or a C₁-C₅ substituted alkyl, specifically, Q may be CH₃ or CH₂CH₃. In one embodiment where Z is compound (V), R₁₃ is independently H, OH or halogen, Q is compound (III).

In one embodiment, a compound of the present disclosure having the general formula (VII) is described where B is NH₂, E is CR₁₅R₁₆ or N, where R₁₅ and R₁₆ are H or a C₁-C₅ unsubstituted alkyl or a C₁-C₅ substituted alkyl, and T is compound (II). In one embodiment when T is compound (II), W is CR₇R₉ with R₇ and R being a H or a C₁-C₅ unsubstituted alkyl or a C₁-C₅ substituted alkyl, R₅ is a H or a C₁-C₅ unsubstituted alkyl or a C₁-C₅ substituted alkyl, and Y is CH₂R₉, with R₉ being a H or a C₁-C₅ unsubstituted alkyl or a C₁-C₅ substituted alkyl, X is phenyl, 3-chlorophenyl, 4-chlorophenyl, 4-fluorophenyl, 3-methylphenyl, 4-methylphenyl, benzyl, hydroxyethyl, fluoroethyl, naphthyl, methyl and ethyl, specifically, X may be phenyl, 3-methylphenyl or 4-chlorophenyl. In an alternate embodiment when Z is compound (II), W is CR₇R₈ with R₇ and R₉ being a H or a C₁-C₅ unsubstituted alkyl or a C₁-C₅ substituted alkyl, R₅ is a H or a C₁-C₅ unsubstituted alkyl or a C₁-C₈ substituted alkyl, and Y is CH₂R₉, with R₉ being a H or a C₁-C₅ unsubstituted alkyl or a C₁-C₅ substituted alkyl, X is R₆S, with R₆ being a H or a C₁-C₅ unsubstituted alkyl or a C₁-C₈ substituted alkyl, specifically R₆ may be CH₃ or CH₂CH₃. In yet another alternate embodiment when Z is compound (II), W is CR₇R₉ with R₇ and R₉ being a H or a C₁-C₅ unsubstituted alkyl or a C₁-C₅ substituted alkyl, R₅ is a H or a C₁-C₅ unsubstituted alkyl or a C₁-C₈ substituted alkyl, and Y is CH₂R₉, with R₉ being a H or a C₁-C₅ unsubstituted alkyl or a C₁-C₈ substituted alkyl, X is H or a C₁-C₅ unsubstituted alkyl or a C₁-C₅ substituted alkyl, specifically, X may be CH₃ or CH₂CH₃. In still another embodiment when Z is compound (II), W is CR₇R₈ with R₇ and R₈ being a H or a C₁-C₅ unsubstituted alkyl or a C₁-C₅ substituted alkyl, R₅ is a H or a C₁-C₅ unsubstituted alkyl or a C₁-C₅ substituted alkyl, and Y is CH₂R₉, with R₉ being a H or a C₁-C₈ alkyl or a C₁-C₈ substituted alkyl, X is compound (III).

In one embodiment, a compound of the present disclosure having the general formula (VII) is described where B is NH₂, E is CR₁₅R₁₆ or N, where R₁₅ and R₁₆ are H or a C₁-C₅ unsubstituted alkyl or a C₁-C₅ substituted alkyl, and T is compound (IV). In one embodiment when T is compound (IV), R₁₀ and R₁ are each independently H, OH or halogen, U is phenyl, 3-chlorophenyl, 4-chlorophenyl, 4-fluorophenyl, 3-methylphenyl, 4-methylphenyl, benzyl, hydroxyethyl, fluoroethyl, naphthyl, methyl and ethyl, specifically, U may be phenyl, 3-methylphenyl or 4-chlorophenyl. In one embodiment where Z is compound (IV), R₁₀ and R₁₁ are each independently H, OH or halogen, U is SR₁₂, with R₁₂ being a H or a C₁-C₅ unsubstituted alkyl or a C₁-C₅ substituted alkyl, specifically R₁₂ may be CH₃ or CH₂CH₃. In one embodiment where Z is compound (I), R₁₀ and R₁₁ are each independently H, OH or halogen, U is H or a C₁-C₅ unsubstituted alkyl or a C₁-C₅ substituted alkyl, specifically, U may be CH₃ or CH₂CH₃. In one embodiment where Z is compound (IV), R₁₀ and R₁₁ are each independently H, OH or halogen, U is compound (III).

In one embodiment, a compound of the present disclosure having the general formula (VII) is described where B is NH₂, E is CR₁₅R₁₆ or N, where R₁₅ and R₁₆ are H or a C₁-C₅ unsubstituted alkyl or a C₁-C₅ substituted alkyl, and T is compound (V). In one embodiment when T is compound (V), R₁₃ is independently H, OH or halogen, Q is phenyl, 3-chlorophenyl, 4-chlorophenyl, 4-fluorophenyl, 3-methylphenyl, 4-methylphenyl, benzyl, hydroxyethyl, fluoroethyl, naphthyl, methyl and ethyl, specifically, Q may be phenyl, 3-methylphenyl or 4-chlorophenyl. In one embodiment where Z is compound (V), R₁₃ is independently H, OH or halogen, Q is SR₁₄, with R₁₄ being a H or a C₁-C₅ unsubstituted alkyl or a C₁-C₅ substituted alkyl, specifically R₁₄ may be CH₃ or CH₂CH₃. In one embodiment where Z is compound (V), R₁₃ is independently H, OH or halogen, Q is H or a C₁-C₅ unsubstituted alkyl or a C₁-C₅ substituted alkyl, specifically, Q may be CH₃ or CH₂CH₃. In one embodiment where Z is compound (V), R₁₃ is independently H, OH or halogen, Q is compound (III).

### Exemplary Synthesis

The following exemplary synthesis illustrates the synthesis of one of the classes of compounds disclosed in the instant specification.

The compound 1 [2-Amino-4-[5-(4-amino-5H-pyrrolo[3,2-d pyrimidin-7-yl)-3,4-dihydroxy-pyrrolidin-2-ylmethylsulfanyl]-butyric acid], is illustrated generally as compound no. 5 in the Exemplary Structures section. The synthesis of compound 1 described below is exemplary of the methods that may be used to and is not meant to exclude other methods of synthesis. As would be known to one of ordinary skill in the art, the synthesis described below may be modified for commercial production.

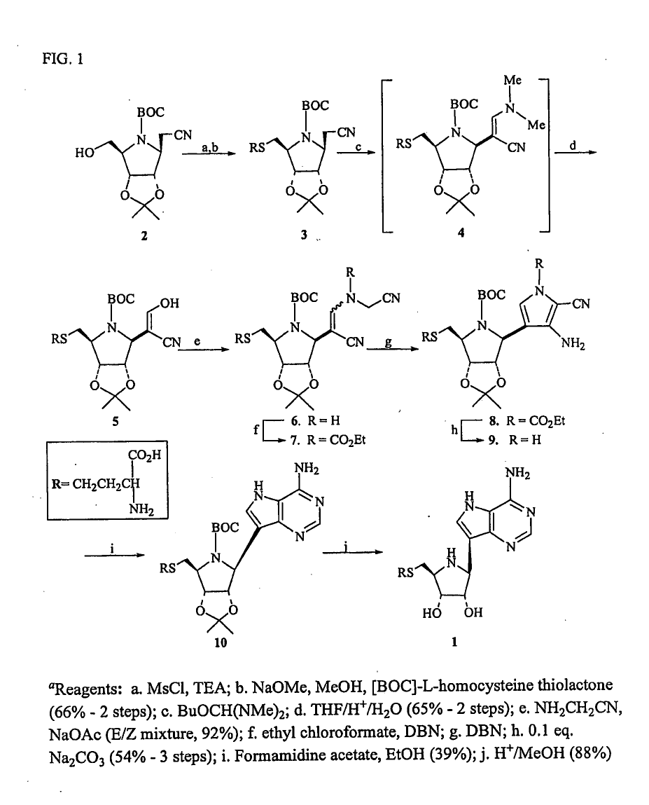

In the generally synthesis, illustrated in FIG. 1, compound 2 (15.5 g, 49.8 mmol) was dissolved in dry CH₂Cl₂ (300 mL) followed by the addition of triethylamine (52.0 mL, 37.3 mmol) and a catalytic amount of DMAP (0.12 g, 0.99 mmol). Methanesulfonyl chloride (5.80. mL g, 74.7 mmol) was then slowly added and the reaction mixture was stirred for 1 h. The mixture was washed with H₂O (2×300 mL), dried, filtered and evaporated to give syrup. The crude sample was purified by column chromatography (7:3 hexanes/EtOAc) to furnish the desired product (18.3 g, 94%) as syrup.

The sample was re-dissolved in MeOH (300 mL) followed by addition of 25% solution of sodium methoxide (21.3 mL, 93.5 mmol) and tert-butoxy carbonyl protected L-homocysteine (16.6 g, 76.8 mmol). The reaction mixture was stirred for 20 h at 60° C. The reaction mixture was neutralized with glacial acetic acid, and the solvent was removed under reduced pressure to give a syrupy residue. The crude sample was purified by column chromatography (7:3 hexanes/EtOAc) to furnish 3 [4-(3-tert-Butoxycarbonylamino-3-methoxycarbonyl-propylsulfanylmethyl)-6-cyanomethyl-2,2-dimethyl-tetrahydro-[1,3]dioxolo[4,5-c]pyrrole-5-carboxylic acid tert-butyl ester] (8.22 g, 32%) as syrup. ¹H NMR (300 MHz, CDCl₃) spectra for compound 3 was determined as: δ 4.80-4.60 (m, 2H), 4.00-4.50 (m, 4H), 3.77 (s, 3H), 2.50-2.82 (m, 4H), 2.12 (s, 2H), 1.45-1.60 (m, 24H). Molecular weight for compound 3 (C₂₅H₄₁N₃O₈S) was calculated as 566.2506 and determined by high resolution mass spectrometry (HRMS) (M+Na)₊ as 566.2516.

Compound 3 (7.87 g, 14.5 mmol) was dissolved in dry DMF (70 mL) followed by addition of tert-butoxy-bis(N,N-dimethylamino)methane (10.6 ml, 50.7 mmol). The reaction was heated to 70° C. for 1 h. Toluene (250 mL) was added and the reaction mixture was washed with H₂O (2×200 mL), dried, filtered and evaporated to give compound 4. The crude sample was re-dissolved in THF/acetic acid/H₂O (1:1:1v/v/v, 120 mL) and stirred at ambient temperature for 4 h. The reaction mixture was extracted with CHCl₃ (250 mL), washed with H₂O (2×300 mL), saturated NaHCO₃ (300 mL), dried, and then evaporated to syrup. The crude sample was purified by column chromatography (7:3 hexanes/EtOAc) to furnish compound 5 [4-(3-tert-Butoxycarbonylamino-3-methoxycarbonyl-propylsulfanylmethyl)-6-(1-cyano-2-hydroxy-vinyl)-2,2-dimethyl-tetrahydro-[1,3]dioxolo[4,5-c]pyrrole-5-carboxylic acid tert-butyl ester] as syrup (5.36 g, 65%-2 steps). ¹H NMR (300 MHz, CDCl₃) spectra for the compound 5 was determined as: δ 7.18 (s, 1H), 4.80-5.00 (m, 2H), 4.40 (m, 1H), 4.05 (dd, 1H), 3.75 (s, 3H), 3.71 (m, 1H), 2.75 (dd, 1H), 2.58-2.70 (m, 3H), 1.90-2.20 (m, 2H), 1.30-1.60 (m, 24H). Molecular weight for compound 5 (C₂₆H₄₁N₃O₉S) was calculated as 594.2455 and determined by high resolution mass spectrometry (HRMS) as 594.2466.

Compound 5 (5.36 g, 93.9 mmol) was dissolved in MeOH (100 mL) followed by addition of aminoacetonitrile (5.21 g, 56.3 mol) and sodium acetate (7.70 g, 93.9 mmol). The reaction mixture was stirred at ambient temperature for 20 h. The solvent was evaporated to dryness and the crude was chromatographed using hexane/EtOAc (1:1) as eluent. The desired fractions were pooled together to furnish 6 [4-(3-tert-Butoxycarbonylamino-3-methoxycarbonyl-propylsulfanylmethyl)-6-[1-cyano-2-(cyanomethyl-amino)-vinyl]-2,2-dimethyl-tetrahydro-[1,3]dioxolo[4,5-c]pyrrole-5-carboxylic acid tert-butyl ester) (5.25 g, 92%) as a mixture E/Z diastereoisomers. The mixture was taken directly to the next step. Molecular weight for compound 6 (C₂₈H₄₃N₅O₈S) was calculated as 632.2724 and determined by high resolution mass spectrometry (HRMS) as 632.2751.

Compound 6 (0.79 g, 1.30 mmol) was dissolved in dry CH₂Cl₂ (25 mL) followed by addition of 1,5-diazabicyclo[4.3.0]non-5-ene (DBN, 0.32 mL, 2.60 mmol) and ethyl chloroformate (0.18 mL, 1.95 mmol). The reaction was stirred at 0° C. for 1 h. TLC of the reaction mixture (toluene/EtOAc, 3:2) showed complete consumption of starting material to give 7. The reaction was removed from the cold bath and allowed to warm to room temperature. 1,5-diazabicyclo[4.3.0]non-5-ene (0.32 mL, 2.60 mmol) was added and the reaction continued for 20 h. The solvent was evaporated to dryness and the crude product 8 was re-dissolved in MeOH (20 mL), 0.1 eq. solid Na₂CO₃ was added and the mixture was stirred for 1 h. TLC (toluene/EtOAc; 3:2) indicated complete conversion of 8 to 9. The solvent was evaporated to dryness and the crude was purified by column chromatography (1:1 hexanes/EtOAc) to give 9 [4-(4-Amino-5-cyano-1H-pyrrol-3-yl)-6-(3-tert-butoxycarbonylamino-3-methoxycarbonyl-propylsulfanylmethyl)-2,2-dimethyl-tetrahydro-[1,3]dioxolo[4,5-c]pyrrole-5-carboxylic acid tert-butyl ester] (0.42 g, 54%-3 steps). ¹H NMR (300 MHz, CDCl₃) spectra for compound 9 was determined as: δ 8.00 (bs, 1H), 6.8 (d, 1H), 4.70-5.05 (m, 5H), 4.00-4.50 (m, 4H), 3.75 (s, 3H), 2.52-2.70 (m, 2H), 1.20-1.55 (m, 24H). Molecular weight for compound 9 (C₂₈H₄₃N₅O₈S) was calculated as 632.2724 and determined by high resolution mass spectrometry (HRMS) as 632.2723.

To a solution of 9 (0.42 g, 0.70 mmol) in EtOH (20 mL), formamidine acetate (0.44 g, 4.21 mmol) was added and the reaction mixture was refluxed for 20 h. The solvent was removed under reduced pressure and chromatographed using (CH₂Cl₂/MeOH, 95:5) to give compound 10 [4-(4-Amino-5H-pyrrolo[3,2-d]pyrimidin-7-yl)-6-(3-tert-butoxycarbonylamino-3-methoxycarbonyl-propylsulfanylmethyl)-2,2-dimethyl-tetrahydro-[1,3]dioxolo[4,5 c]pyrrole-5-carboxylic acid tert-butyl ester] (0.17 g, 39%). ¹H NMR (300 MHz, CDCl₃) spectra for compound 10 was determined as: δ 8.15 (bs, 3H), 7.60 (s, 1H), 7.45 (s, 1H), 7.45 (s, 1H), 5.23 (s, 2H), 4.81 (t, 1H), 4.00-4.30 (m, 3H), 3.65 (s, 1H), 2.55 (m, 2H), 2.00 (s, 3H), 1.2-1.5 (m, 24H). Molecular weight for compound 10 (C₂₉H₄₄N₆O₈S) was calculated as 637.3014 and determined by high resolution mass spectrometry (HRMS) as 637.2997.

Compound 10 (0.17 g, 0.27 mmol) was dissolved in MeOH (5 mL) and 0.5N sodium hydroxide solution and the reaction was refluxed at 75° C. for 2 h. Upon completion of the reaction the solvent was evaporated to dryness. The crude mixture was then redissolved in MeOH (20 mL) and concentrated HCl (3 mL) and then heated to 50° C. for 1 h. The solvent was evaporated to dryness and the residue was co-evaporated with EtOH (2×25) to give a white powder. The residue was re-dissolved in H₂O (2 mL), and then passed through a HPLC column. The appropriate fractions were pooled together to give a solid residue. The residue was dissolved in H₂O (8 mL) and filtered through a Millipore filter (0.25) and lyophilized to give a white solid, 1 [2-Amino-4-[5-(4-amino-5H-pyrrolo[3,2-d]pyrimidin-7-yl)-3,4-dihydroxy-pyrrolidin-2-ylmethylsulfanyl]-butyric acid] (88 mg, 88%). ¹H NMR (300 MHz, D₂O) spectra for compound 1 was determined as: δ 8.24 (s, 1H), 7.85 (s, 1H), 4.87 (t, 2H), 4.43 (t, 1H), 3.87 (m, 1H), 3.82 (m, 1H), 3.00-3.30 (m, 2H), 2.80 (t, 2H), 2.20 (m, 2H). ¹³C NMR spectra for compound 1 was determined as: δ 174.4, 150.5, 147.4, 131.1, 113.8, 106.8, 73.4, 72.9, 63.6, 57.1, 54.1, 31.7, 30.6, 27.3, 8.8. Molecular weight for 1 was calculated as 383.1496 as determined by high resolution mass spectrometry (HRMS) as 383.1504.

### Enzyme Assay and Inhibitor Analysis:

The gene for E. coli MTA/AdoHcy nucleosidase (MTAN) was amplified and cloned into a bacterial hexahistidine plasmid expression vector. Induced recombinant enzyme was purified by Nickel-chelate chromatography, and dialyzed into 100 mM sodium phosphate buffer, pH 7. Enzyme purity was assessed by SDS-PAGE and concentration assigned using the experimentally determined extinction coefficient (ε₂₈₀=10480 M⁻¹cm⁻¹).

MTAN assays were performed in 1 mL quartz cuvettes using a Perkin Elmer Lambda 35 UV/V is spectrometer outfitted with KinLab and UV WinLab software. This direct UV spectrophotometric assay follows the decrease in absorbance at 275 nm that accompanies the cleavage of MTA to MTR and Ade. (2-(4-Amino-5H-pyrrolo[3,2-d]pyrimidin-7-yl)-5-methyl-sulfanylmethyl-pyrrolidine-3,4-diol) and (2-Amino-4-[5-(4-amino-5H-pyrrolo[3,2-d]pyrimidin-7-yl)-3,4 dihydroxy-pyrrolidin-2-ylmethylsulfanyl]-butyric acid) inhibitor concentrations were assigned using UV absorbance and the extinction coefficient of 9-deazaadenine. Reaction conditions consisted of 100-250 μM MTA, 50 mM sodium phosphate (pH 7), and 0-1 μM inhibitor at 25° C. Reactions were initiated by the addition of 90 μmol MTAN enzyme to the 1 mL assay mixture, and the change in UV absorbance followed for 20-25 minutes. Controls consisted of reactions containing no inhibitor or no enzyme. Inhibitor constants for the initial rate reactions (Kᵢ's) were calculated by fitting the results to the equation for competitive inhibition. Since the inhibitors showed reaction kinetics consistent with the model of slow-onset, tight binding transition state inhibitors, a second inhibitor dissociation constant (Kᵢ*) describing this conformational tightening of the enzyme-inhibitor complex could be calculated using the steady state reaction rates (measured at 15-20 minutes in the reaction) and the equation for competitive inhibition. The observed Ki and Kᵢ*values for these compounds are less than 10 nM.

### Pharmaceutical Compositions, Modes of Administration and Methods of Treatment

The present disclosure provides compounds of the general formula (I), (VI) and (VII) as detailed above which are inhibitors of MTAN and/or MTAP. Both eukaryotic and prokaryotic forms of MTAN and/or MTAP may be inhibited by the compounds disclosed. The compounds of the present disclosure are therefore expected to have clinical utility in treating and/or preventing a variety of disease states and/or conditions related to the expression of MTAN and/or MTAP. For example, such disease states and conditions may include cancer, and those caused by or related to bacterial infections and protozoan parasitic infections.

The present disclosure provides for a pharmaceutical composition comprising a pharmaceutically effective amount of a at least one compound of general formula (I), (VI) and (VIII) as described herein. Such pharmaceutical compositions may be used in the manufacture of a medicament for treating and/or preventing a disease or condition in which it is desirable to inhibit MTAN and/or MTAP. Such pharmaceutical compositions and medicaments may also comprise a pharmaceutically acceptable carrier. The compounds of the disclosure are useful in both free form and in the form of pharmaceutically acceptable salts.

The pharmaceutically acceptable carriers described herein, including, but not limited to, vehicles, adjuvants, excipients, or diluents, are well-known to those who are skilled in the art. Typically, the pharmaceutically acceptable carrier is chemically inert to the active compounds and has no detrimental side effects or toxicity under the conditions of use. The pharmaceutically acceptable carriers can include polymers and polymer matrices.

The compounds described in the instant disclosure can be administered by any conventional method available for use in conjunction with pharmaceuticals, either as individual therapeutic agents or in combination with additional therapeutic agents.

The compounds described are administered in pharmaceutically effective amount. The pharmaceutically effective amount of the compound and the dosage of the pharmaceutical composition administered will, of course, vary depending upon known factors, such as the pharmacodynamic characteristics of the particular agent and its mode and route of administration; the age, health and weight of the recipient; the severity and stage of the disease state or condition; the kind of concurrent treatment; the frequency of treatment; and the effect desired.

A daily dosage of active ingredient can be expected to be about 0.001 to 1000 milligrams (mg) per kilogram (kg) of body weight. In one embodiment, the total amount is between about 0.1 mg/kg and about 1000 mg/kg of body weight; in an alternate embodiment between about 1.1 mg/kg and about 100 mg/kg of body weight; in yet another alternate embodiment between 0.1 mg/kg and about 30 mg/kg of body weight. The above described amounts may be administered as a series of smaller doses over a period of time if desired. As would be obvious, the dosage of active ingredient may be given other than daily if desired.

The total amount of the compound administered will also be determined by the route, timing and frequency of administration as well as the existence, nature, and extent of any adverse side effects that might accompany the administration of the compound and the desired physiological effect. It will be appreciated by one skilled in the art that various conditions or disease states, in particular chronic conditions or disease states, may require prolonged treatment involving multiple administrations.

Dosage forms of the pharmaceutical compositions described herein (forms of the pharmaceutical compositions suitable for administration) contain from about 0.1 mg to about 500 mg of active ingredient (i.e. the compounds disclosed) per unit. In these pharmaceutical compositions, the active ingredient will ordinarily be present in an amount of about 0.5-95% weight based on the total weight of the composition. Multiple dosage forms may be administered as part of a single treatment.

The active ingredient can be administered orally in solid dosage forms, such as capsules, tablets, and powders, or in liquid dosage forms, such as elixirs, syrups and suspensions. It can also be administered parenterally, in sterile liquid dosage forms. The active ingredient can also be administered intranasally (nose drops) or by inhalation via the pulmonary system, such as by propellant based metered dose inhalers or dry powders inhalation devices. Other dosage forms are potentially possible such as administration transdermally, via patch mechanism or ointment.

Formulations suitable for oral administration can consist of (a) liquid solutions, such as a pharmaceutically effective amount of the compound dissolved in diluents, such as water, saline, or orange juice; (b) capsules, sachets, tablets, lozenges, and troches, each containing a predetermined pharmaceutically effective amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, propylene glycol, glycerin, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, in addition to the active ingredient, such carriers as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the patient, and aqueous and non-aqueous sterile suspensions that can include suspending agents, solubilizers, thickening agents, stabilizers, and preservatives. The compound can be administered in a physiologically acceptable diluent in a pharmaceutically acceptable carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyldialkylammonium halides, and alkylpyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkyl .beta.-aminopropionates, and 2-alkylimidazoline quaternary ammonium salts, and (e) mixtures thereof.

The parenteral formulations typically contain from about 0.5% to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

Pharmaceutically acceptable excipients are also well-known to those who are skilled in the art. The choice of excipient will be determined in part by the particular compound, as well as by the particular method used to administer the composition. Accordingly, there is a wide variety of suitable formulations of the pharmaceutical composition of the present invention. The following methods and excipients are merely exemplary and are in no way limiting. The pharmaceutically acceptable excipients preferably do not interfere with the action of the active ingredients and do not cause adverse side-effects. Suitable carriers and excipients include solvents such as water, alcohol, and propylene glycol, solid absorbants and diluents, surface active agents, suspending agent, tableting binders, lubricants, flavors, and coloring agents.

The compounds of the present invention, alone or in combination with other suitable components, can be made into aerosol formulations to be administered via inhalation. These aerosol formulations can be placed into pressurized acceptable propellants, such as dichlorodifluoromethane, propane, and nitrogen. Such aerosol formulations may be administered by metered dose inhalers. They also may be formulated as pharmaceuticals for non-pressured preparations, such as in a nebulizer or an atomizer.

The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets. The requirements for effective pharmaceutically acceptable carriers for injectable compositions are well known to those of ordinary skill in the art. See Pharmaceutics and Pharmacy Practice, J.B. Lippincott Co., Philadelphia, Pa., Banker and Chalmers, Eds., 238-250 (1982) and ASHP Handbook on Injectable Drugs, Toissel, 4th ed., 622-630 (1986).

Formulations suitable for topical administration include pastilles comprising the active ingredient in an inert base, such as gelatin and glycerin, or sucrose and acacia, as well as creams, emulsions, and gels containing, in addition to the active ingredient, such carriers as are known in the art.

Additionally, formulations suitable for rectal administration may be presented as suppositories by mixing with a variety of bases such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

One skilled in the art will appreciate that suitable methods of administering a compound of the present invention to an patient are available, and, although more than one route can be used to administer a particular compound, a particular route can provide a more immediate and more effective reaction than another route.

In one embodiment, the teachings of the present disclosure provide for the use of such pharmaceutical compositions and medicaments in a method of treating a disease state and/or condition in which it is desired to inhibit or reduce the activity of MTAN and/or MTAP. The method of treatment comprises the steps of providing such pharmaceutical composition containing at least one compound of the general formula (I), (VI) and (VII) and administering such pharmaceutical composition in a therapeutically effective amount to inhibit or reduce the activity of MTAN and/or MTAP in a patient in need of such treatment.

In one embodiment, the teachings of the present disclosure provide for the use of such pharmaceutical compositions and medicaments in a method of preventing or suppressing a disease state and/or condition in which it is desired to inhibit or reduce the activity of MTAN and/or MTAP. The method of preventing or suppressing comprises the steps of providing such pharmaceutical composition containing at least one compound of the general formula (I), (VI) and (VII) and administering such pharmaceutical composition in a therapeutically effective amount to inhibit or reduce the activity of MTAN and/or MTAP in a patient in need of such treatment.

The compounds and pharmaceutical compositions of the present disclosure can be administered to patients to prevent and/or treat a number of cancers. Cancers include, but are not limited to, leukemias and lymphomas such as acute lymphocytic leukemia, acute nonlymphocytic leukemias, chronic lymphocytic leukemia, chronic myelogenous leukemia, Hodgkin's Disease, non-Hodgkin's lymphomas, and multiple myeloma, childhood solid tumors such as brain tumors, neuroblastoma, retinoblastoma, Wilms Tumor, bone tumors, and soft-tissue sarcomas, common solid tumors of adults such as lung cancer, colon and rectum cancer, breast cancer, prostate cancer, urinary cancers, uterine cancers, oral cancers, pancreatic cancer, melanoma and other skin cancers, stomach cancer, ovarian cancer, brain tumors, liver cancer, laryngeal cancer, thyroid cancer, esophageal cancer, and testicular cancer. As discussed above, methods of treatment for treating patients with such cancers are also within the scope of this disclosure. In one embodiment the compounds and pharmaceutical compositions disclosed are used in such prevention and/or treatment methods to inhibit MTAP.

The compounds and pharmaceutical compositions of the present disclosure can be administered to prevent and/or treat a number of bacterial infections and conditions caused by or related thereto, including both gram-positive and gram negative bacterial infections. Exemplary bacteria that may cause a human disease state or condition that may be treated by the compounds and pharmaceutical compositions disclosed herein include, but are not limited to, Legionella species, Campylobacter species, Staphylococcus species, E. coli species, Borrelia species, Helicobacter species, Ehrlichia species, Clostridium species, Vibrio species, Bartonella species, Streptococcus species, Chlamydia species, Clostridium species, Neisseria species, Pseudomonas species, Xanthomonas species, Agrobacterium species, Brucella species, Francisella species, Vibrio species, Acinetobacter species, Haemophilus species, Salmonella species, Yersinia species, Bacillus species, Corynebacterium species, Mycobacterium, species, Chlamydia species, Mycoplasma species, Klebsiella species, Salmonella species, Leptospirosis species, Fusobacterium species, Listeria species, Proteus species, Bacteroides species, and Porphyromonas species. In one embodiment the compounds and pharmaceutical compositions disclosed are used in such prevention and/or treatment methods to inhibit MTAN.

The methods of the treating and preventing also comprises further administering of a chemotherapeutic agent in combination with and of the compounds or pharmaceutical compositions of the present disclosure. Any suitable chemotherapeutic agent can be employed for this purpose. The chemotherapeutic agent is typically selected from the group consisting of alkylating agents, antimetabolites, natural products, hormonal agents, and miscellaneous agents.

Examples of alkylating chemotherapeutic agents include carmustine, chlorambucil, cisplatin, lomustine, cyclophosphamide, melphalan, mechlorethamine, procarbazine, thiotepa, uracil mustard, triethylenemelamine, busulfan, pipobroman, streptozocin, ifosfamide, dacarbazine, carboplatin, and hexamethylmelamine.

Examples of chemotherapeutic agents that are antimetabolites include cytosine arabinoside, fluorouracil, gemcitabine, hydroxyurea, mercaptopurine, methotrexate, azaserine, thioguanine, floxuridine, fludarabine, cladribine and L-asparaginase.

Examples of chemotherapeutic agents that are natural products include actinomycin D, bleomycin, camptothecins, daunomycin, doxorubicin, etoposide, mitomycin C, TAXOL (paclitaxel), taxotere, teniposide, vincristine, vinorelbine, mithramycin, idarubicin, MITHRACIN™ (plicamycin), and deoxycoformycin.

An example of a hormonal chemotherapeutic agent includes tamoxifen. Examples of the aforesaid miscellaneous chemotherapeutic agents include mitotane, mitoxantrone, vinblastine, and levamisole.

Useful pharmaceutical dosage forms for administration of the compounds according to the present invention can be illustrated as follows:

A large number of unit capsules are prepared by filling standard two-piece hard gelatine capsules each with 100 mg of powdered active ingredient, 150 mg of lactose, 50 mg of cellulose and 6 mg of magnesium stearate.

A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.

A large number of tablets are prepared by conventional procedures so that the dosage unit is 100 mg of active ingredient, 0.2 mg of colloidal silicon dioxide, 5 mg of magnesium stearate, 275 mg of microcrystalline cellulose, 11 mg of starch, and 98.8 mg of lactose. Appropriate aqueous and non-aqueous coatings may be applied to increase palatability, improve elegance and stability or delay absorption.

Immediate release tablets/capsules are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

Moreover, the compounds of the present invention can be administered in the form of nose drops, or metered dose and a nasal or buccal inhaler. The drug is delivered from a nasal solution as a fine mist or from a powder as an aerosol.

The foregoing description illustrates and describes the compounds of the present disclosure. Additionally, the disclosure shows and describes only the preferred embodiments of the compounds but, as mentioned above, it is to be understood that the teachings of the present disclosure are capable of use in various other combinations, modifications, and environments and is capable of changes or modifications within the scope of the inventive concept as expressed herein, commensurate with the above teachings and/or the skill or knowledge of the relevant art. The embodiments described hereinabove are further intended to explain best modes known of practicing the invention and to enable others skilled in the art to utilize the invention in such, or other, embodiments and with the various modifications required by the particular applications or uses of the invention. Accordingly, the description is not intended to limit the invention to the form disclosed herein. All references cited herein are incorporated by reference as if fully set forth in this disclosure.

1. A compound having the general formula (I): 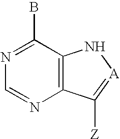
wherein,
A is selected from the group consisting of N and CD, where D is selected from the group consisting of: H, halogen, unsubstituted alkyl, substituted alkyl, optionally substituted aryl, optionally substituted heterocycle, optionally substituted cycloalkyl, OH, NH₂, NHR₁, NR₁R₂ and SR₃;
B is selected from the group consisting of NH₂ and NHR₄;
R₁, R₂, R₃, and R₄, each independently selected from the group consisting of: H, unsubstituted alkyl, substituted alkyl, optionally substituted heterocycle, optionally substituted cycloalkyl and optionally substituted aryl;
Z is selected from a compound having the general formula: 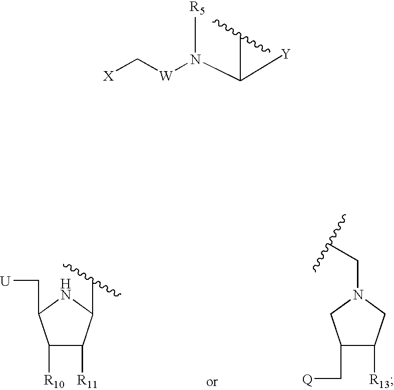
or a tautomer thereof; or a polymorph thereof, or a pharmaceutically acceptable salt thereof; or an ester thereof; or a prodrug thereof.

2. The compound of claim 1 where Z is 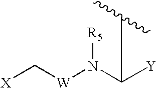
wherein,
W is selected from the group consisting of CHR₇ and CR₇R₈,
Y is selected from the group consisting of H and CH₂R₉;
X is selected from the group consisting of: R₆S, H, unsubstituted alkyl, substituted alkyl, optionally substituted heterocycle, optionally substituted cycloalkyl, optionally substituted aryl and 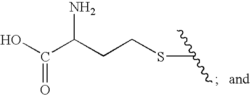
R₅, R₆, R₇, R₈ and R₉ are each independently selected from the group consisting of: H, unsubstituted alkyl, substituted alkyl, optionally substituted heterocycle, optionally substituted cycloalkyl and optionally substituted aryl, provided that when X is R₆S, R₆ is not CH₃.

3. The compound of claim 2 where X is

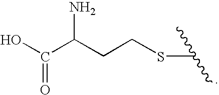

4. The compound of claim 2 where X is compound III, B is NH₂, A is CH, R₅ is H, Y is CH₃ and W is CH₂.

5. The compound of claim 1 which is 2-Amino-4-[5-(4-amino-5H-pyrrolo[3,2-d]pyrimidin-7-yl)-3,4-dihydroxy-pyrrolidin-2-ylmethylsulfanyl]-butyric acid, or a tautomer thereof; or a polymorph thereof, or a pharmaceutically acceptable salt thereof; or an ester thereof; or a prodrug thereof.

6. A compound which is 2-Amino-4-[5-(4-amino-5H-pyrrolo[3,2-d]pyrimidin-7-yl)-3,4-dihydroxy-pyrrolidin-2-ylmethylsulfanyl]-butyric acid.

7. The compound of claim 2 where X is selected from the group consisting of phenyl, 3-chlorophenyl, 4-chlorophenyl, 4-fluorophenyl, 3-methylphenyl, 4-methylphenyl, benzyl, hydroxyethyl, fluoroethyl, naphthyl, methyl and ethyl.

8. The compound of claim 2 where X is R₆S and R₆ is selected from the group consisting of H, unsubstituted alkyl, substituted alkyl, optionally substituted heterocycle, optionally substituted cycloalkyl and optionally substituted aryl, provided said unsubstituted alkyl group is not CH₃.

9. The compound of claim 2 where X is R₆S and R₆ is selected from the group consisting of H, C₁-C₅ unsubstituted alkyl and C₁-C₅ substituted alkyl.

10. The compound of claim 2 where X is R₆S and R₆ is CH₂CH₃.

11. The compound of claim 2 where X is selected from the group consisting of H, C₁-C₅ unsubstituted alkyl and C₁-C₅ substituted alkyl.

12. The compound of claim 2 where X is CH₃ or CH₂CH₃.

13. The compound of claim 1 where Z is 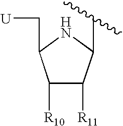
wherein,
U is selected from the group consisting of: R₁₂S, H, unsubstituted alkyl, substituted alkyl, optionally substituted heterocycle, optionally substituted cycloalkyl, optionally substituted aryl and 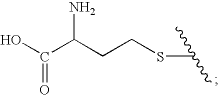
R₁₂ is selected from the group consisting of H, unsubstituted alkyl, substituted alkyl, optionally substituted heterocycle, optionally substituted cycloalkyl and optionally substituted aryl; and
R₁₀ and R₁₁ are each independently selected from the group consisting of H, OH and halogen.

14. The compound of claim 13 where U is selected from the group consisting of phenyl, 3-chlorophenyl, 4-chlorophenyl, 4-fluorophenyl, 3-methylphenyl, 4-methylphenyl, benzyl, hydroxyethyl, fluoroethyl, naphthyl, methyl and ethyl.

15. The compound of claim 13 where U is R₁₂S and R₁₂ is selected from the group consisting of H, unsubstituted alkyl, substituted alkyl, optionally substituted heterocycle, optionally substituted cycloalkyl and optionally substituted aryl, provided said unsubstituted alkyl group is not CH₃.

16. The compound of claim 13 where U is R₁₂S and R₁₂ is selected from the group consisting of a H, C₁-C₅ unsubstituted alkyl and C₁-C₅ substituted alkyl, provided said unsubstituted alkyl group is not CH₃.

17. The compound of claim 13 where U is R₁₂S and R₁₂ is CH₂CH₃.

18. The compound of claim 13 where U is selected from the group consisting of H, C₁-C₅ unsubstituted alkyl and C₁-C₅ substituted alkyl.

19. The compound of claim 13 where U is CH₃ or CH₂CH₃.

20. The compound of claim 13 where U is

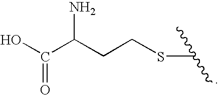

21. The compound of claim 13 where U is 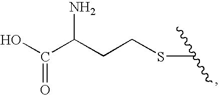
B is NH₂, A is CH, and R₁₀ and R₁₁ are each OH.

22. The compound of claim 1 where Z is 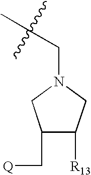
wherein,
Q is selected from the group consisting of: R₁₄S, H, unsubstituted alkyl, substituted alkyl, optionally substituted heterocycle, optionally substituted cycloalkyl, optionally substituted aryl and 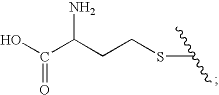
R₁₄ is selected from the group consisting of H, unsubstituted alkyl, substituted alkyl, optionally substituted heterocycle, optionally substituted cycloalkyl and optionally substituted aryl; and
R₁₃ is selected from the group consisting of H, OH and halogen.

23. The compound of claim 22 where Q is selected from the group consisting of phenyl, 3-chlorophenyl, 4-chlorophenyl, 4-fluorophenyl, 3-methylphenyl, 4-methylphenyl, benzyl, hydroxyethyl, fluoroethyl, naphthyl, methyl and ethyl.

24. The compound of claim 22 where Q is R₁₄S and R₁₄ is selected from the group consisting of H, unsubstituted alkyl, substituted alkyl, optionally substituted heterocycle, optionally substituted cycloalkyl and optionally substituted aryl, provided said unsubstituted alkyl group is not CH₃.

25. The compound of claim 22 where Q is R₁₄S and R₁₄ is selected from the group consisting of H, C₁-C₅ unsubstituted alkyl and C₁-C₅ substituted alkyl, provided said unsubstituted alkyl group is not CH₃.

26. The compound of claim 22 where Q is R₁₄S and R₁₄ is CH₂CH₃.

27. The compound of claim 22 where Q is selected from the group consisting of H, C₁-C₅ unsubstituted alkyl and C₁-C₅ substituted alkyl.

28. The compound of claim 22 where Q is CH₃ or CH₂CH₃.

29. The compound of claim 22 where Q is

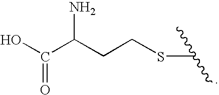

30. The compound of claim 22 where Q is 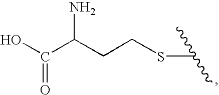
B is NH₂, A is CH, and R₁₃ is OH.

31-86. (canceled)

87. A method for treating a disease or condition in which it is desirable to inhibit methylthioadenosine phosphorylase (MTAP) in a subject in need of said treatment, said treatment comprising administering to the subject a therapeutically effective amount of a compound of claim 1 where said compound inhibits MTAP.

88. (canceled)

89. The method of claim 87 where said disease state or condition is a cancer.

90. The method of claim 89 where the cancer is selected from the group consisting of: leukemias and lymphomas such as acute lymphocytic leukemia, acute nonlymphocytic leukemias, chronic lymphocytic leukemia, chronic myelogenous leukemia, Hodgkin's Disease, non-Hodgkin's lymphomas, and multiple myeloma, childhood solid tumors such as brain tumors, neuroblastoma, retinoblastoma, Wilms Tumor, bone tumors, and soft-tissue sarcomas, common solid tumors of adults such as lung cancer, colon and rectum cancer, breast cancer, prostate cancer, urinary cancers, uterine cancers, oral cancers, pancreatic cancer, melanoma and other skin cancers, stomach cancer, ovarian cancer, brain tumors, liver cancer, laryngeal cancer, thyroid cancer, esophageal cancer, and testicular cancer.

91. A method according to claim 87 further comprising administering to said subject a therapeutic agent.

92. The method of claim 91 where said therapeutic agent is selected from the group consisting of: an alkylating agent, an antimetabolite, a natural product, and a hormonal agent.

93. (canceled)

94. The method of claim 99 where said disease state or condition is a bacterial infections or a condition related thereto.

95. The method of claim 94 where the bacterial infection is caused by a gram negative or a gram positive bacteria.

96. A pharmaceutical composition comprising a compound of claim 1 and a pharmaceutically acceptable carrier or diluent.

97. A compound which is 2-(4-Amino-5H-pyrrolo[3,2-d]pyrimidin-7-yl)-5-methyl-sulfanylmethyl-pyrrolidine-3,4-diol and which is an inhibitor of MTAN, or a polymorph thereof, or a pharmaceutically acceptable salt thereof; or an ester thereof; or a prodrug thereof.

98. (canceled)

99. A method for treating a disease or condition in which it is desirable to inhibit 5′-methylthioadenosine/S-adenosylhomocysteine nucleosidase (MTAN) in a subject in need of said treatment, said treatment comprising administering to the subject a therapeutically effective amount of a compound of claim 1 where said compound inhibits MTAN.

100. The method of claim 99 where said compound is 2-(4-Amino-5H-pyrrolo[3,2-d]pyrimidin-7-yl)-5-methyl-sulfanylmethyl-pyrrolidine-3,4-diol or a polymorph thereof, or a pharmaceutically acceptable salt thereof; or an ester thereof; or a prodrug thereof.
